(19) United States
(12) Patent Application Publication
(10) Pub. No.: US 2026/0233183 A1
(43) Pub. Date: Aug. 13, 2026

(54) CATALYTIC PROCESS USING AMMONIA AS HYDROGEN SOURCE FOR CARBON DIOXIDE CONVERSION

(71) Applicant: Agency for Science, Technology and Research, Singapore (SG)

(72) Inventors: Mingyao Kelvin KWOK, Jurong Island (SG); Luwei CHEN, Jurong Island (SG); Chee Kok POH, Jurong Island (SG); Sin Kim TANG, Jurong Island (SG)

(21) Appl. No.: 19/475,145

(22) PCT Filed: Apr. 20, 2023
(86) PCT No.: PCT/SG2023/050269

§ 371 Date: Oct. 14, 2025

(51) Int. Cl. B01J 8/02; B01J 23/42; B01J 23/46; B01J 23/745; B01J 23/75; B01J 23/755; B01J 37/03; B01J 37/16; C01B 3/326; C01B 32/40; C07C 1/02

(52) U.S. Cl. CPC B01J 8/0285; B01J 23/42; B01J 23/462; B01J 23/745; B01J 23/75; B01J 23/755; B01J 37/03; B01J 37/16; C01B 3/326; C01B 32/40; C07C 1/02; B01J 2208/00017; B01J 2523/821; B01J 2523/828; B01J 2523/842; B01J 2523/845; B01J 2523/847; C01B 2203/062; C01B 2203/1058; C01B 2203/107; C01B 2203/1082; C01B 2203/1205

(57) ABSTRACT

The invention relates to a process and an apparatus for producing syngas and/or hydrocarbons. The process involves reacting ammonia and carbon dioxide in a single reactor in the presence of a transition metal catalyst to form one or more products comprising carbon monoxide, hydrogen and one or more hydrocarbons. The invention allows green ammonia as a liquefied hydrogen carrier to be used directly in CO2 conversion.

### FIELD OF THE INVENTION

The invention relates to a process for preparing one or more reaction products from ammonia and carbon dioxide, where the reaction products are selected from carbon monoxide, one or more C₁₋₃ hydrocarbons, and hydrogen.

### BACKGROUND TO THE INVENTION

Syngas (also known as synthesis gas) is a mixture of hydrogen and carbon monoxide, which sometimes also contains carbon dioxide and methane, which may be present at varying ratios.

Syngas is industrially useful in the production of a number of important chemicals, such as synthetic fuels, ammonia fertilisers, polymer monomers, and methanol. Furthermore, syngas is also used as an intermediate for hydrogen production through the water gas shift reaction (1) below.

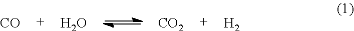

Current industrial methods for the preparation of syngas include steam methane reforming, or coal gasification as shown in reactions (2) and (3) below.

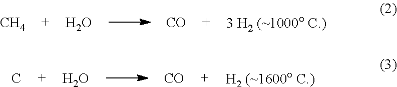

However, these reactions are endothermic, so process heating and off-gases result in high greenhouse gas emissions that contribute a large amount to the 1.5 gigatons of CO₂ emissions from industrial chemical industry. Furthermore, synthetic fuels produced from fossil-derived syngas result in further CO₂ emissions. Therefore, there is a need for the preparation of syngas, and its downstream products, from non-fossil fuel sources. The development of such a process would have a large role to play in reducing future CO₂ emissions.

There are two main approaches to producing syngas directly from CO₂, both of which are neither at commercial megaton/year scale nor net-zero. First, reverse water gas shift (RWGS) reacts CO₂ with excess hydrogen to produce CO (reaction (4) below), which CO is then blended with additional hydrogen to obtain the appropriate composition for downstream applications (typically a H₂/CO ratio of 2-3), while water is removed by condensation. In this case, if the hydrogen is not obtained from net-zero sources (e.g. electrolysis powered by carbon-free electricity), there would still be net CO₂ emissions. Second, dry reforming of methane reacts CO₂ with CH₄ to produce a theoretical 1:1 mixture of CO and H₂ (reaction (5) below), which is then similarly blended with additional hydrogen. Here, the conversion of a methane feedstock into CO would result in net CO₂ emissions from the downstream uses of the CO.

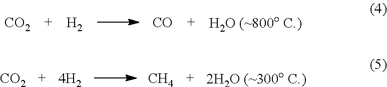

Therefore, hydrogenation of CO₂ to form syngas is a potentially viable method for producing net-zero syngas (and its downstream chemicals), so long as hydrogen is obtained in a carbon-free manner. For example, “green” hydrogen can be obtained from electrolysis of water powered by solar/wind/nuclear energy. However, hydrogen is very difficult to store—at standard conditions it has extremely low density (0.0899 kg/m³), while liquid H₂ (71 kg/m³) suffers from high liquefaction costs and boil-off rates on the order of 1% per day. The flammability/explosiveness of hydrogen also causes safety concerns during storage and transport, increasing costs.

Ammonia is a promising liquid carrier of hydrogen, with high hydrogen density, high density when liquefied, and relatively high temperature liquefaction (−33° C.). Ammonia may be transported much more efficiently, easily and safely than liquid hydrogen. Green ammonia may also be obtained commercially from multiple sources.

However, when used as a hydrogen carrier, ammonia must be cracked to regenerate hydrogen before use, as shown in reaction (6) below.

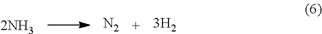

Current methods for cracking ammonia are energy-intensive and cost-ineffective due their small scales and the high cost of purifying, storing, and transporting the resulting hydrogen. This is especially the case for industries that require high hydrogen purity, such as fuel cells. Even if the resulting hydrogen is to be used in reactions where the nitrogen is inert, a very large ammonia cracking plant must be provided on-site for the production of even relatively low amounts of hydrogen, which increases site footprint and reduces economic viability as compared to existing methane reforming (which is not green). Furthermore, since ammonia conversion and CO₂ conversion occur under different process conditions (e.g. temperature and pressure), and are conducted using different catalysts, the use of ammonia as a hydrogen source for the preparation of syngas is still a costly multi-stage process.

There is a need for a simplified process that solves some or all of the problems associated with existing methods for the production of syngas/hydrocarbons.

### SUMMARY OF THE INVENTION

Despite the concerns regarding purifying and storing hydrogen in the art, the presence of nitrogen in the environment is not a concern for reactions (4) and (5) above. Therefore, in the production of syngas, ammonia may be cracked into hydrogen and nitrogen, and the subsequent gas stream used directly in the production of syngas in reactions (4) and (5) above.

The current inventors have surprisingly found that this may be done as an integrated process in a single reactor using a single catalyst. Thus, the invention may convert ammonia (NH₃) and carbon dioxide (CO₂) into an outlet stream containing, depending on the catalyst, synthesis gas (syngas, containing carbon monoxide and hydrogen) and/or hydrocarbons such as methane. Examples of reactions that may occur are provided as reactions (7) and (8) below.

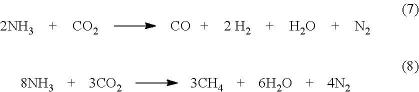

Thus, the invention allows green ammonia as a liquefied hydrogen carrier to be used directly in CO₂ conversion, which advantageously reduces the cost of separately regenerating, purifying, storing, and transporting hydrogen (H₂) gas. The process may advantageously be used as a greener alternative to the production of fossil- and hydrocarbon-derived syngas. Green syngas prepared by the invention may be used to decarbonise downstream chemical applications such as synthetic fuels and industrial chemicals, while green hydrocarbons can be used as a substitute to fossil fuels during the green fuel transition.

Therefore, the invention provides the following.
1. A process for producing one or more reaction products, the process comprising:
   (i) providing ammonia and carbon dioxide to a reactor comprising a transition metal catalyst; and
   (ii) reacting the ammonia and carbon dioxide in the presence of the transition metal catalyst to form one or more reaction products selected from the group consisting of carbon monoxide, one or more C₁₋₃ hydrocarbons, and hydrogen, wherein step (ii) is conducted in a single reactor.
2. The process according to Clause 1, wherein the transition metal catalyst comprises a transition metal from one or more of Groups 8 to 11 of the periodic table.
3. The process according to Clause 1 or 2, wherein the transition metal catalyst comprises one or more transition metals selected from the group consisting of Co, Fe, Cu, Ni, Ru and Pt, optionally wherein the transition metal catalyst comprises Co.
4. The process according to any one of the preceding clauses, wherein the transition metal catalyst is in the form of a solid metal catalyst.
5. The process according to any one of the preceding clauses, wherein the transition metal catalyst is in the form of a solid metal catalyst provided on a solid support.
6. The process according to Clause 5, wherein the solid support comprises one or more of the group consisting of silica, alumina, carbon, ceria, zirconia, gallium oxide, indium oxide, and magnesium oxide, optionally wherein the solid support comprises silica.
7. The process according to any one of the preceding clauses, wherein the transition metal catalyst is obtainable by a process comprising:
   (a) co-precipitation of a transition metal salt and a solid support precursor; and
   (b) calcining or reducing the resulting product, optionally wherein the solid support comprises silica and the solid support precursor comprises tetraethylorthosilicate.
8. The process according to Clause 5, or according to Clause 6 or 7 as dependent on Clause 5, wherein the transition metal catalyst comprises from 1 wt. % to 70 wt. % transition metal.
9. The process according to any one of the preceding clauses, wherein the molar ratio of ammonia:carbon dioxide is from about 0.67:1 to about 10:1, optionally from about 1:1 to about 4:1.
10. The process according to any one of the preceding clauses, wherein step (ii) is conducted at a temperature of from about 250° C. to about 1000° C., optionally from about 300° C. to about 700° C., more optionally from about 400° C. to about 650° C., such as about 500° C. to about 600° C.
11. The process according to Clause 10, wherein step (ii) is conducted at pressure of from atmospheric pressure to about 3,000 kPa, optionally from about atmospheric pressure to about 500 kPa.
12. The process according to any one of the preceding clauses, the process further comprising:
   (iii) cooling the output products from step (ii) in a gas-liquid separator to provide a gaseous stream comprising one or more of carbon monoxide, one or more C₁₋₃ hydrocarbons, and hydrogen,
   and optionally subjecting the gaseous stream to a separation process to remove at least a portion of any gaseous nitrogen present in the gaseous stream.
13. The process according to Clause 12, wherein step (iii) is conducted at a temperature of from about 1° C. to about 40° C., optionally from about 10° C. to about 30° C., such as about 25° C.
14. The process according to Clause 12 or 13, wherein step (iii) also provides a liquid stream comprising one or more of ammonium carbonate, ammonium bicarbonate and ammonium carbamate, and where the process further comprises:
   (iv) passing the liquid stream comprising one or more of ammonium carbonate, ammonium bicarbonate and ammonium carbamate through an evaporator to regenerate ammonia and carbon dioxide.
15. The process according to Clause 14, wherein step (iv) is conducted at a temperature of from about 40° C. to about 80° C., such as about 60° C.
16. The process according to any one of the preceding clauses, wherein step (ii) is conducted in an atmosphere comprising less than 1 vol. % O₂, optionally less than 0.1 vol. % O₂, more optionally less than 0.01 vol. % O₂.
17. The process according to any one of the preceding clauses, comprising one or both of the following precursor steps:
   (A) obtaining a gaseous stream comprising carbon dioxide from a carbon capture process or from a stock of liquid carbon dioxide; and
   (B) obtaining a gaseous stream comprising ammonia from a stock of liquid ammonia.
18. The process according to any one of the preceding clauses, wherein step (ii) is conducted in a reactor that is heated by a source selected from a fuel (e.g. natural gas or ammonia), electrical heating or waste heat.
19. The process according to any one of the preceding clauses, wherein the one or more C₁₋₃ hydrocarbons comprises one or more of the group consisting of methane, ethane, ethene, and propane,
   optionally wherein the one or more C₁₋₃ hydrocarbons comprises methane.
20. The process according to any one of the preceding clauses, wherein:
   the one or more C₁₋₃ hydrocarbons comprises methane;
   the transition metal catalyst comprises one or more transition metals selected from the group consisting of Co, Fe, Cu, Ni, Ru and Pt in the form of a solid metal catalyst provided on a solid support; and
   step (ii) is conducted at a temperature of from about 300° C. to about 700° C.
21. The process according to Clause 20, the process further comprising:
   (iii) cooling the output products from step (ii) in a gas-liquid separator to provide a gaseous stream comprising one or more of carbon monoxide, one or more C₁₋₃ hydrocarbons, and hydrogen, and a liquid stream comprising one or more of ammonium carbonate, ammonium bicarbonate and ammonium carbamate; and
   (iv) passing the liquid stream comprising one or more of ammonium carbonate, ammonium bicarbonate and ammonium carbamate through an evaporator to regenerate ammonia and carbon dioxide,
   optionally wherein step (iii) comprises subjecting the gaseous stream to a separation process to remove at least a portion of any gaseous nitrogen present in the gaseous stream.
22. The process according to any one of the preceding clauses, wherein the transition metal catalyst comprises from 0.1 wt. % to 20 wt. % of an alkali metal,
optionally wherein the alkali metal is selected from one or more of the group consisting of lithium, sodium, potassium and rubidium,
more optionally wherein the alkali metal is selected from one or more of the group consisting of sodium and potassium.
23. An apparatus for conducting the process according to any one of Clauses 1 to 22, wherein the apparatus comprises:
   (a) a carbon dioxide storage tank for storing carbon dioxide in liquid form;
   (b) an ammonia storage tank for storing ammonia in liquid form;
   (c) a packed bed catalytic reactor comprising a transition metal catalyst in the form of a solid metal provided on a solid support, said packed bed catalytic reactor being suitable for converting carbon dioxide and ammonia into one or more of carbon monoxide and methane;
   (d) a thermal energy source for heating the packed bed catalytic reactor;
   (e) a gas-liquid separator; and
   (f) an evaporator.

### BRIEF DESCRIPTION OF THE FIGURES

FIG. 1 shows steps in the production of green syngas.

FIG. 2 shows the NH₃ cracking ability of various catalysts.

FIG. 3 shows the results of a>340 h long experiment with Co@SiO₂ catalyst.

FIG. 4 shows thermogravimetric analysis (TGA) of a spent Co@SiO₂ catalyst.

FIG. 5 shows X-ray diffraction results for urea, ammonium carbamate, ammonium carbonate, ammonium bicarbonate, and solids obtained using the process of the invention.

### DETAILED DESCRIPTION OF THE INVENTION

The invention provides a process for producing one or more reaction products, the process comprising:
(i) providing ammonia and carbon dioxide to a reactor comprising a transition metal catalyst; and
(ii) reacting the ammonia and carbon dioxide in the presence of the transition metal catalyst to form one or more reaction products selected from the group consisting of carbon monoxide, one or more C₁₋₃ hydrocarbons, and hydrogen, wherein step (ii) is conducted in a single reactor.

As used herein, the term “ammonia” may be used interchangeably with its chemical formula, “NH₃”. Likewise, the term “carbon dioxide” may be used interchangeably with its chemical formula “CO₂”, the term “carbon monoxide” may be used interchangeably with its chemical formula “CO”. The chemical formula “H₂” may be used to indicate molecular hydrogen, e.g. gaseous hydrogen.

The invention also provides an apparatus for conducting the process according to the invention, wherein the apparatus comprises:
(a) a carbon dioxide storage tank for storing carbon dioxide in liquid form;
(b) an ammonia storage tank for storing ammonia in liquid form;
(c) a packed bed catalytic reactor comprising a transition metal catalyst in the form of a solid metal provided on a solid support, said packed bed catalytic reactor being suitable for converting carbon dioxide and ammonia into one or more of carbon monoxide and methane;
(d) a thermal energy source for heating the packed bed catalytic reactor;
(e) a gas-liquid separator; and
(f) an evaporator.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

As used herein, the singular forms “a,” “an,” and “the” include plural referents unless the context clearly dictates otherwise. Thus, for example, reference to “a composition” includes mixtures of two or more such compositions, reference to “a metal” includes mixtures of two or more such metals, and the like.

The one or more reaction products generated by the process of the invention are selected from the group consisting of carbon monoxide, one or more C₁₋₃ hydrocarbons, and hydrogen. As will be appreciated by a person skilled in the art, the balance of reaction products may be altered by changing the process conditions (e.g. catalyst, temperature, ammonia:carbon dioxide ratio). Thus, the process of the invention may be used to produce syngas, one or more hydrocarbons (such as methane), or to produce both syngas and hydrocarbons. In this context, a skilled person will understand that references herein to the production of syngas refer to the production of a gas that comprises both carbon monoxide and hydrogen, but which may also include hydrocarbons such as methane.

The process of the invention may provide one or more C₁₋₃ hydrocarbons. As will be appreciated by a person skilled in the art, the reaction conditions may provide one or more hydrocarbons selected from the group consisting of methane, ethane, ethene, and propane. In particular embodiments of the invention that may be mentioned herein, the one or more C₁₋₃ hydrocarbons may comprise methane.

The invention comprises a step of providing ammonia and carbon dioxide to a reactor comprising a transition metal catalyst. The ammonia and carbon dioxide may be provided to a reactor as a single combined stream, or as two separate streams. Since both of these gases will be allowed to mix in the reactor itself, it does not matter whether they are mixed prior to entry to the reactor. The molar ratio of ammonia to carbon dioxide may be adjusted as required, and is not particularly limited. In some embodiments of the invention that may be mentioned herein, the molar ratio of ammonia:carbon dioxide may be from about 0.67:1 to about 10:1, such as from about 1:1 to about 4:1.

For the avoidance of doubt, the ammonia and carbon dioxide used in the process of the invention may, or may not, be obtained from “green”, renewable, or environmentally friendly sources. The origin of these gases is not important to the process of the invention, and the process of the invention may be performed with ammonia and carbon dioxide obtained from any source. Nevertheless, an advantage of the invention is that it enables the preparation of syngas and/or hydrocarbons from green feedstocks.

As used herein, the term “about” may mean that a value may deviate by up to 20% from the stated amount. For example, the value may deviate by up to 20%, 18%, 15%, 13%, 10%, 9%, 8%, 7%, 6%, or 5%.

The invention subsequently involves a step of reacting the ammonia and carbon dioxide in the presence of the transition metal catalyst to form one or more reaction products selected from the group consisting of carbon monoxide, one or more C₁₋₃ hydrocarbons, and hydrogen (i.e. syngas and/or hydrocarbons). This step is explained in more detail below.

NH₃ and CO₂ gases may be combined into a heated reactor (e.g. a packed bed reactor) containing a solid powder catalyst containing one or more transition metals. The one or more transition metals may be unsupported or supported on one or more inert catalyst carriers. At high temperatures, the catalyst cracks NH₃ to provide hydrogen gas, which reacts with CO₂ to form syngas and/or hydrocarbons, with side products containing water and nitrogen. Water can be condensed, while unreacted ammonia and carbon dioxide can be captured in aqueous solution as ammonium carbonate/bicarbonate or ammonium carbamate for recycling. Trace ammonia can be captured by adsorption, in order to obtain a high-purity outlet gas stream. By tuning the catalyst and reaction conditions, high selectivity for either syngas or methane can be obtained.

The combination of CO₂ with NH₃ as a direct feedstock to a syngas/methanation reactor, together with a suitable catalyst and process conditions for syngas/methane production, allows for the following features and advantages.
a) Combining NH₃ cracking and CO₂ hydrogenation into an integrated single reactor to produce syngas and methane, resulting in improved cost and process efficiency. In a traditional two-reactor series, one reactor would crack NH₃ into H₂, with costly H₂ purification and storage processes in between, and the second reactor would react CO₂ with H₂ to form syngas and methane. Hence, this invention would save capital and operational costs of one reactor (compared to two) and the intermediary costs of purifying and storing H₂.
b) NH₃ is used as a reducing agent rather than H₂, allowing for higher energy density and more efficient storage of feedstock, as NH₃ is much easier to liquefy than H₂.
c) Zero NOx production from the usage of NH₃ in this process. In comparison, if NH₃ is used to produce electricity from turbines to power a turbine for the generation of H₂ from electrolysis or direct electrochemical CO₂ reduction, significant amount of NOx pollutant could be produced.
d) Simple separation/purification process for removal and recycling of unreacted NH₃ and CO₂. If the two processes of NH₃ cracking and CO₂ hydrogenation are separated, the H₂ from NH₃ needs to be purified by pressure swing adsorption, while any unreacted CO₂ needs to be purified by amine adsorption. However, in this invention both unreacted NH₃ and CO₂ will be automatically captured in the outlet liquid water stream as ammonium bicarbonate.
e) Catalyst enables new production pathway of useful chemicals from NH₃ and CO₂. Existing technologies for reacting both NH₃ and CO₂ are non-catalytic and only produce urea at high temperatures and pressures. This invention produces syngas and methane instead using catalysts.

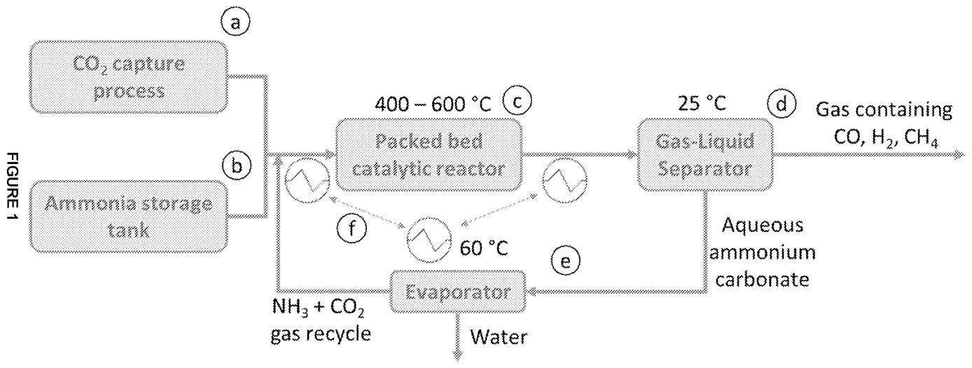

Steps involved in the green preparation of syngas/methane from ammonia and carbon dioxide are shown in FIG. 1.
A CO₂ feed may be obtained from CO₂ capture as a gas (a), while ammonia (b) may be stored in liquid form and re-gasified before entering the reactor (c).
The catalyst used in (c) may be in the form of solid powders or pellets.
The reactor is typically thermally powered, with the source of heating being flexible, e.g. fuels such as natural gas or ammonia, electrical heating, or waste heat.
The outlet hot gases may be cooled in a gas-liquid separator (d), which removes product water and unreacted CO₂ and NH₃ as aqueous ammonium carbonate/bicarbonate. The outlet gas thus consists of gases selected from CO, H₂, CH₄, inert N₂ and CO₂ and trace NH₃ that can be utilized directly in downstream applications, or purified further if necessary.
The aqueous ammonium carbonate may pass through an evaporator (e) to release NH₃ and CO₂ gas that can be pumped back into the reactor feed for recycling, while wastewater can be disposed of.

The process of the invention uses a transition metal catalyst. In some embodiments of the invention that may be mentioned herein, the transition metal catalyst may comprise a transition metal from one or more of Groups 8 to 11 of the periodic table. For example, the transition metal catalyst may comprise one or more transition metals selected from the group consisting of Co, Fe, Cu, Ni, Ru and Pt. In particular embodiments of the invention, the transition metal catalyst may comprise Co.

In some embodiments of the invention that may be mentioned herein, the transition metal catalyst may be provided in the form of a solid metal catalyst, i.e. in the form of the metal itself in oxidation state 0. As will be appreciated by a person skilled in the art, the catalyst will typically be provided in a powder, pellet, or high surface area supported form in order to maximise the surface area available for reactions to occur.

Therefore, in some embodiments of the invention that may be mentioned herein, the transition metal catalyst may be in the form of a solid metal catalyst provided on a solid support. Suitable solid supports include any material known to function as an inert catalyst support under the reaction conditions of the process of the invention. Thus, in some embodiments of the invention that may be mentioned herein, the solid support may comprise one or more of the group consisting of silica, alumina, carbon, ceria, zirconia, gallium oxide, indium oxide, and magnesium oxide. In particular embodiments of the invention, the solid support may comprise silica. Typically, the transition metal catalyst may be in the form of a powder. In some embodiments, the catalyst may have a surface area in the range of, for example, about 50 to about 400 m²/g.

The transition metal catalyst may be obtainable by a process that comprises:
(a) co-precipitation of a transition metal salt and a solid support precursor; and
(b) calcining or reducing the resulting product.

In some embodiments of the invention, the resulting product from step (a) or (b) may be ground to a powder. Therefore, in some embodiments of the invention the transition metal catalyst may be in the form of a powder. The transition metal catalyst may comprise the transition metal present within a solid support matrix.

When the solid support comprises silica, the solid support precursor may comprise tetraethylorthosilicate.

When the transition metal catalyst comprises a solid support, the transition metal catalyst may comprise any suitable amount of transition metal and any suitable amount of solid support. For example, in some embodiments of the invention that may be mentioned herein, the transition metal catalyst may comprise from 1 wt. % to 70 wt. % transition metal.

The transition metal catalyst may further comprise one or more alkali metals. Without being bound by theory, it is believed that the inclusion of an alkali metal will help to increase the adsorption of CO₂, which is an acidic gas, onto the catalyst surface during the process of the invention. Thus, in some embodiments of the invention that may be mentioned herein, the transition metal catalyst may comprise from 0.1 wt. % to 20 wt. % of an alkali metal. Suitable alkali metals that may be mentioned herein include lithium, sodium, potassium and rubidium (e.g. sodium and potassium).

Step (ii) of the process of the invention may be conducted at any suitable temperature. For example, in some embodiments of the invention that may be mentioned herein, step (ii) may be conducted at a temperature of from about 250° C. to about 1000° C. In further embodiments of the invention that may be mentioned herein, step (ii) may be conducted at a temperature of from about 300° C. to about 700° C. In further embodiments of the invention that may be mentioned herein, step (ii) may be conducted at a temperature of from about 400° C. to about 650° C. In further embodiments of the invention that may be mentioned herein, step (ii) may be conducted at a temperature of from about 500° C. to about 600° C.

For the avoidance of doubt, any end point of any range mentioned herein may be combined with any other end point from any other range in respect of the same variable. Thus, for the temperatures above, the following ranges are explicitly contemplated herein.
From about 250° C. to about 300° C., from about 250° C. to about 400° C., from about 250° C. to about 500° C., from about 250° C. to about 600° C., from about 250° C. to about 700° C., from about 250° C. to about 1000° C.;
from about 300° C. to about 400° C., from about 300° C. to about 500° C., from about 300° C. to about 600° C., from about 300° C. to about 700° C., from about 300° C. to about 1000° C.;
from about 400° C. to about 500° C., from about 400° C. to about 600° C., from about 400° C. to about 700° C., from about 400° C. to about 1000° C.;
from about 500° C. to about 600° C., from about 500° C. to about 700° C., from about 500° C. to about 1000° C.;
from about 600° C. to about 700° C., from about 600° C. to about 1000° C.; and
from about 700° C. to about 1000° C.

Step (ii) of the process of the invention may be conducted at any suitable pressure. For example, in some embodiments of the invention that may be mentioned herein, step (ii) may be conducted at a pressure of from atmospheric pressure to about 3,000 kPa. In further embodiments of the invention that may be mentioned herein, step (ii) may be conducted at a pressure of from about atmospheric pressure to about 500 kPa.

The process of the invention may further comprise:
(iii) cooling the output products from step (ii) in a gas-liquid separator to provide a gaseous stream comprising one or more of carbon monoxide, one or more C₁₋₃ hydrocarbons, and hydrogen.

This cooling step (iii) may be conducted at any suitable temperature. For example, in some embodiments of the invention that may be mentioned herein, step (iii) may be conducted at a temperature of from about 1° C. to about 40° C. In further embodiments of the invention that may be mentioned herein, step (iii) may be conducted at a temperature of from about 10° C. to about 30° C. In further embodiments of the invention that may be mentioned herein, step (iii) may be conducted at a temperature of about 25° C.

The resulting gaseous stream from step (iii) may further be passed through a separation process to remove at least a portion of any gaseous nitrogen present in the gaseous stream.

The cooling step (iii) may also provide a liquid (e.g. aqueous) stream comprising one or more of ammonium carbonate, ammonium bicarbonate and ammonium carbamate. Thus, the process may further comprise:
(iv) passing the liquid stream comprising one or more of ammonium carbonate, ammonium bicarbonate and ammonium carbamate through an evaporator to regenerate ammonia and carbon dioxide.

The evaporation step (iv) may be conducted at any suitable temperature. For example, in some embodiments of the invention that may be mentioned herein, step (iv) may be conducted at a temperature of from about 40° C. to about 80° C., such as about 60° C. As will be appreciated by a person skilled in the art, in order to increase the rate of evaporation of liquid (e.g. water) in step (iv), this step may be performed under reduced pressure.

As will be appreciated by a person skilled in the art, step (ii) may advantageously be performed in an atmosphere having a reduced amount of oxygen, e.g. under anaerobic conditions. This may improve the lifetime and efficiency of the catalyst, and also minimise the amount of nitrogen oxides that may otherwise be generated under the temperature and pressure of the reaction. Thus, in some embodiments of the invention that may be mentioned herein, step (ii) may be conducted in an atmosphere comprising less than 1 vol. % O₂. In further embodiments of the invention that may be mentioned herein, step (ii) may be conducted in an atmosphere comprising less than 0.1 vol. % O₂. In further embodiments of the invention that may be mentioned herein, step (ii) may be conducted in an atmosphere comprising less than 0.01 vol. % O₂.

As discussed herein, the process of the invention may provide an advantageously green (i.e. advantageously environmentally friendly) process for the preparation of syngas and/or hydrocarbons. Therefore, the process may comprise a precursor step of obtaining carbon dioxide from a carbon capture process. Alternatively, it may be preferred to use a feedstock of liquid carbon dioxide, which may have been, but is not necessarily, obtained by a carbon capture process.

Thus, in some embodiments of the invention that may be mentioned herein, the process may comprise a precursor step:
(A) obtaining a gaseous stream comprising carbon dioxide from a carbon capture process or from a stock of liquid carbon dioxide.

The process may also include a precursor step of obtaining a gaseous ammonia stream from a stock of liquid ammonia. Thus, in some embodiments of the invention that may be mentioned herein, the process may comprise a precursor step:
(B) obtaining a gaseous stream comprising ammonia from a stock of liquid ammonia.

As discussed herein, the process of the reaction may be conducted at increased temperatures in the reactor (e.g. in the packed bed reactor). Thus, in some embodiments of the invention that may be mentioned herein, step (ii) may be conducted in a reactor that is heated by a source selected from a fuel (e.g. natural gas or ammonia), electrical heating or waste heat.

In a particular embodiment of the invention that may be mentioned herein:
the one or more C₁₋₃ hydrocarbons, when present, may comprise methane;
the transition metal catalyst may comprise one or more transition metals selected from the group consisting of Co, Fe, Cu, Ni, Ru and Pt in the form of a solid metal catalyst provided on a solid support; and
step (ii) may be conducted at a temperature of from about 300° C. to about 700° C.

In a particular example of this embodiment, the invention may comprise the steps:
(iii) cooling the output products from step (ii) in a gas-liquid separator to provide a gaseous stream comprising one or more of carbon monoxide, one or more C₁₋₃ hydrocarbons, and hydrogen, and a liquid stream comprising one or more of ammonium carbonate, ammonium bicarbonate and ammonium carbamate; and
(iv) passing the liquid stream comprising one or more of ammonium carbonate, ammonium bicarbonate and ammonium carbamate through an evaporator to regenerate ammonia and carbon dioxide.

In a specific example of this particular embodiment, step (iii) may comprise subjecting the gaseous stream to a separation process to remove at least a portion of any gaseous nitrogen present in the gaseous stream.

As discussed herein, the invention also provides an apparatus for conducting the process according to the invention, wherein the apparatus comprises:
(a) a carbon dioxide storage tank for storing carbon dioxide in liquid form;
(b) an ammonia storage tank for storing ammonia in liquid form;
(c) a packed bed catalytic reactor comprising a transition metal catalyst in the form of a solid metal provided on a solid support, said packed bed catalytic reactor being suitable for converting carbon dioxide and ammonia into one or more of carbon monoxide and methane;
(d) a thermal energy source for heating the packed bed catalytic reactor;
(e) a gas-liquid separator; and
(f) an evaporator.

The components (a) to (f) may be any suitable component known to a person skilled in the art that is consistent with the disclosure hereinabove.

### EXAMPLES

### Source of Materials:

Co/Fe/Cu/Ni-containing catalysts:
Copper (II) nitrate hemi (pentahydrate), (Cu(NO₃)₂·2.5H₂O, >98%), Alfa Aesar
Nickel (II) nitrate hexahydrate, (Ni(NO₃)₂·6H₂O, >98%), Alfa Aesar
Iron (III) nitrate nonahydrate, (Fe(NO₃)₃·9H₂O, >98%), Sigma-Aldrich Co. Ltd.
Cobalt (II) nitrate hexahydrate, (Co(NO₃)₂·6H₂O, >98%), Sigma-Aldrich Co. Ltd.
Cetrimonium chloride 25 wt. % in H₂O, CTAC, Sigma-Aldrich Co. Ltd.
Sodium hydroxide pellets for analysis (NaOH, >99.9%), Merck KGaA.
Tetraethyl orthosilicate, TEOS (Si(OC₂H₅)₄, >98%), Alfa Aesar.
Ethanol absolute, EtOH, analytical reagent (C₂H₅OH, >99.8%), VWR BDH chemical.
Ru and Pt-containing catalysts:
Ruthenium (III) chloride hydrate, (RuCl₃·XH₂O, >99.8%), Sigma-Aldrich Co. Ltd.
Chloroplatinic acid hexahydrate, (H₂PtCl₆·6H₂O, >37.5% Pt basis), Sigma-Aldrich Co. Ltd.
Sodium hydroxide pellets for analysis (NaOH, >99.9%), Merck KGaA.
Ethylene glycol, (C₂H₆O₂, anhydrous, 99.8%), Sigma-Aldrich Co. Ltd.
Ethanol absolute, EtOH, analytical reagent (C₂H₅OH, >99.8%), VWR BDH chemical.
Hexadecyltrimethylammonium chloride, 25 wt. % in H₂O, CTACI, Sigma-Aldrich Co. Ltd.
Triethanolamine, TEA, GR for analysis (N(CH₂CH₂OH)₃, >99.9%), Merck KGaA.
Tetraethyl orthosilicate, TEOS (Si(OC₂H₅)₄, >98%), Alfa Aesar.

### Preparative Example: Catalyst Synthesis

Metal-doped silica-supported catalysts were prepared by co-precipitation, where all synthesis can be scaled up simply by multiplying the amounts of reagents used. The symbol “M@SiO₂” is used to refer to the developed catalysts, which are supported on SiO₂, and where M is the metal used.

Typically for Co/Fe/Cu/Ni metals, 50 wt % (by metal oxide CuO, Fe₂O₃, Co₃O₄, NiO) of the precursor nitrate salt (i.e. 6.1 g Cu(NO₃)₂·2.5H₂O hemipentahydrate, 10.3 g Fe(NO₃) 3.9H₂O, 7.42 g Co(NO₃)₂·6H₂O or 7.8 g Ni(NO₃)₂·6H₂O is dissolved in a mixture of 3.51 mL 25% CTAC (cetrimonium chloride) in 400 mL water. Then, 80 mL of 1.25M NaOH solution is added under stirring for a few hours. The precipitate is collected by centrifugation, re-dispersed in 320 mL of water, and adjusted to a pH>12 using NaOH solution. Then, a mixture of 7.6 mL TEOS (tetraethylorthosilicate) and 80 mL ethanol is added dropwise and the resultant mixture stirred for 48 h. The final solid is separated by centrifugation, dried overnight at 80° C., and calcined at 500° C. in static air for 2 h.

For Ru and Pt metals, 2 wt % of precursor RuCl₃.XH₂O (0.2 g) or H₂PtCl₆·6H₂O (0.5 g) is added to a mixture of 0.4 mL NaOH and 100 mL ethylene glycol. The solution is heated to 80° C. and held for 30 min for complete dissolution, then heated to 160° C. for 3 h under reflux before cooling back to room temperature to form a colloidal suspension. Then, 75 mL of this colloidal suspension is added to a mixture of 195 mL water, 15 mL ethanol, 30 mL 25% CTACI, and 7.5 mL triethanolamine. Then, 7.5 mL of TEOS was added dropwise under stirring, and the mixture heated at 60° C. for 2 h. The final solid is separated by centrifugation and washing with ethanol, dried at 80° C. overnight, and calcined in N₂ at 400° C. for 6 h.

### General Method 1: Catalytic Process Conditions

In a typical single-pass (i.e. without recycling) reaction run, CO₂ and NH₃ gases are fed via mass flow controllers to a 0.25 inch stainless steel (SS316) tube packed with 200 mg of catalyst, heated by an electric furnace. The catalyst is first reduced under 20 mL/min of pure H₂ flow at 250-600° C. (depending on the metal) for 2 h. Then, the temperature is adjusted to the reaction temperature of 600° C. under the flow of NH₃ first, then CO₂. A ratio of NH₃/CO₂ of from 1 to 4 is used (with an NH₃ flowrate of 20-80 mL/min). An inert gas such as argon can be added for measurement of reaction conversion and product yield. In a single-pass configuration, the aqueous ammonium carbonate is collected in a glass condenser while the outlet gas can be analysed by a gas chromatograph equipped with a thermal conductivity detector (TCD) and a Porapak Q column and a MolSieve 5A column (with appropriate valve switching for both NH₃ and CO₂ to bypass the MolSieve 5A column).

### Working Example

### Catalyst Performance: Cracking of Ammonia

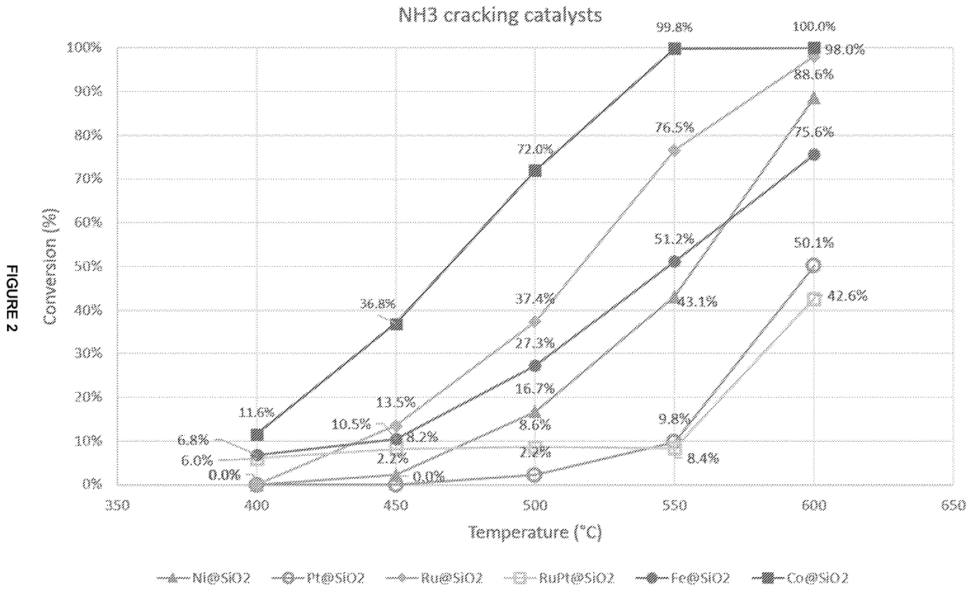

Using steps set out in General Method 1, the single-pass catalysts performance for pure NH₃ cracking (i.e. CO₂/NH₃=0) was first tested at various temperatures to ascertain the catalysts' monofunctional activity, the results are shown in FIG. 2.

### Catalyst Performance: Syngas/Hydrocarbon Production

The single-pass catalyst performance of the various catalysts was then tested for NH₃—CO₂ reaction. The results are summarized in Table 1 below (General Method 1, 200 mg catalyst, flowrate of Ar: NH₃ of 10:20 mL/min, 20 h time-on-stream).

TABLE 1

| | | NH₃/ | | | | | Yield of |
|---|---|---|---|---|---|---|---|
| | Reduction | CO₂ | NH₃ | CO₂ | CH₄ | CO | major |
| Catalyst | temp. (° C.) | ratio | conversion | conversion | selectivity | selectivity | product |
| Ni@SiO₂ | 600 | 4 | 88.7% | 80.4% | 82.3% | 17.7% | 66.2% CH₄ |
| Ni@SiO₂ | 600 | 1 | 74.4% | 37.1% | 45.2% | 54.8% | 20.3% CO |
| Ru@SiO₂ | 400 | 4 | 79.2% | 86.4% | 88.7% | 11.3% | 76.6% CH₄ |
| Ru@SiO₂ | 400 | 1 | 86.8% | 37.9% | 44.5% | 55.5% | 21% CO |
| Fe@SiO₂ | 600 | 4 | 61.4% | 87.2% | 0% | 100% | 87.2% CO |
| Co@SiO₂ | 400 | 4 | 90.8% | 72.3% | 23% | 77% | 55.7% CO |
| Co@SiO₂ | 400 | 2 | 86.6% | 61.3% | 5.7% | 94.3% | 57.8% CO |
| Co@SiO₂ | 400 | 1 | 81.5% | 44.4% | 1.8% | 98.2% | 43.6% CO |
| Pt@SiO₂ | 250 | 4 | 55.7% | 99% | 0% | 100% | 99% CO |

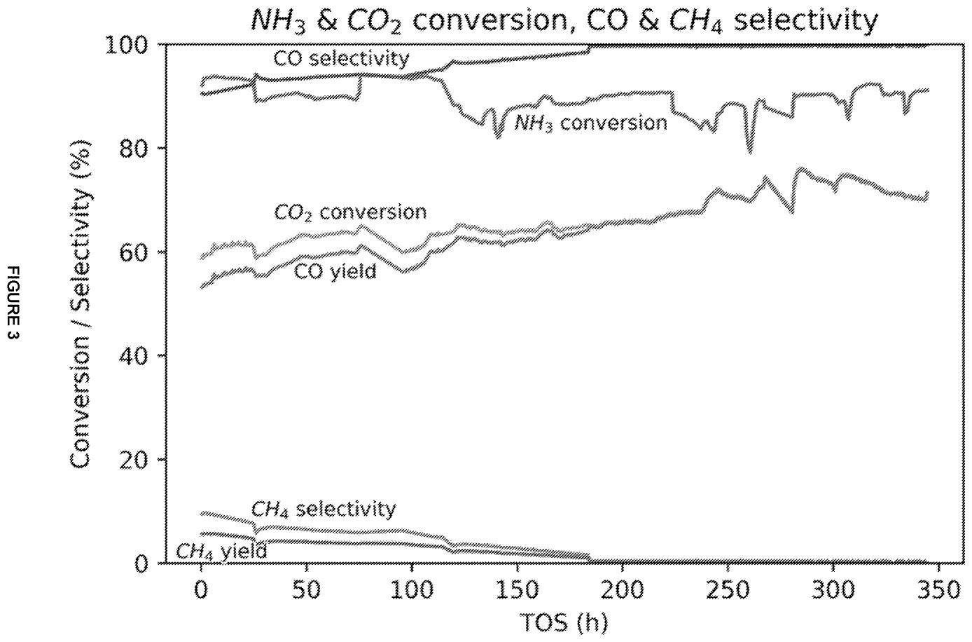

In addition, a>340 h long single-pass experiment with Co@SiO₂ catalyst shows high stability and excellent performance over time, as shown in Table 2 below and FIG. 3. The catalyst was selected as it had a good balance of high NH₃ conversion, CO₂ conversion and CO selectivity, and the highest CO yield achieved was 72.8% and the highest NH₃ conversion was 93.3%. At the flowrate of NH₃: CO₂=40:20 mL/min, this performance corresponds to 77.7 L/day production syngas with H₂/CO ratio of 2.7 (or H₂/[CO+CO₂] ratio of 2).

TABLE 2

| Time-on- | NH₃ | CO₂ | CH₄ | CO | CO |
|---|---|---|---|---|---|
| stream (h) | conversion | conversion | selectivity | selectivity | yield |
| 50 | 89.8% | 63.2% | 6.5% | 93.5% | 59.1% |
| 100 | 93.3% | 60.5% | 5.9% | 94.1% | 56.9% |
| 150 | 87.9% | 63.9% | 2.8% | 97.2% | 62.1% |
| 200 | 90.4% | 65.8% | 0.4% | 99.6% | 65.5% |
| 250 | 88.4% | 70.9% | 0.4% | 99.6% | 70.7% |
| 300 | 90.3% | 73.1% | 0.4% | 99.6% | 72.8% |
| 345 | 91.1% | 71.5% | 0% | 100% | 71.5% |

Additionally, no NOx was detected in any of the reactions, as all reactions were conducted anaerobically (i.e. without oxygen).

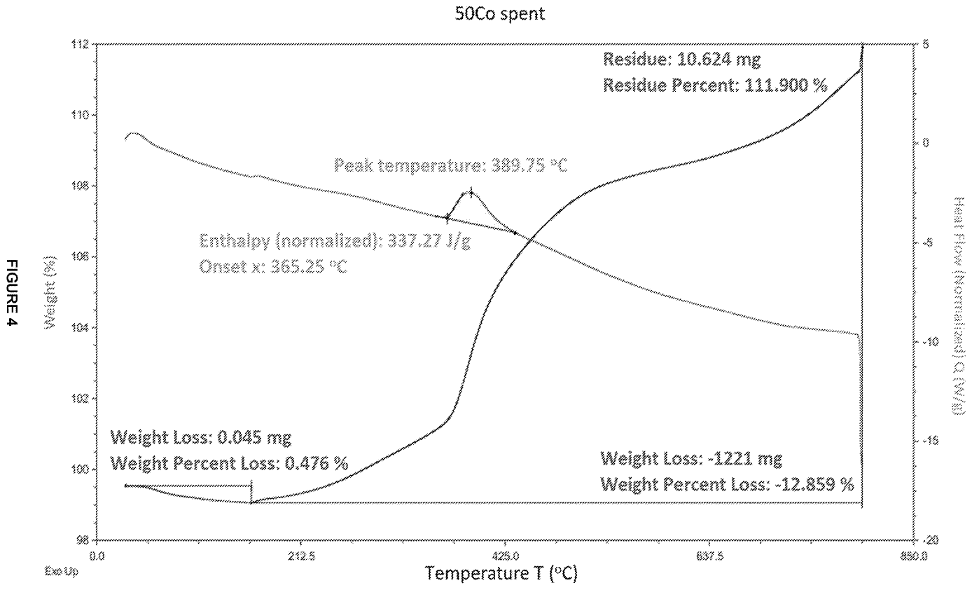

Thermogravimetric analysis (TGA) of the spent Co@SiO₂ catalyst showed no carbon deposition on the catalyst. The only observed changes were 0.5% mass loss below 200° C. attributed to adsorbed water loss and 12.9% mass gain around 350-500° C. which is attributed to re-oxidation of Co metal to oxide. Results are shown in FIG. 4.

### Collection of Ammonium Carbonate/Carbamate for Recycling

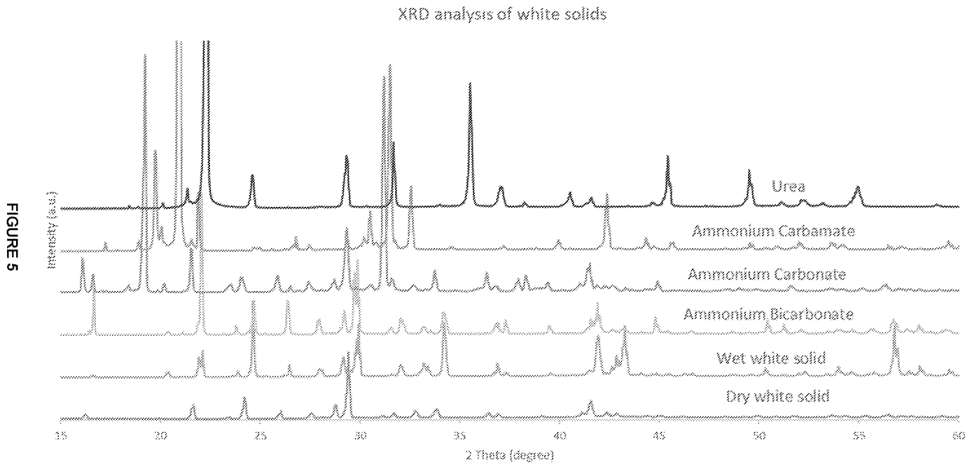

Different white powders could be collected from the reactor outlet through a gas-liquid separator after cooling and drying, depending on whether it was collected in the presence or absence of water. The identity of the powders was analysed by X-ray diffraction (XRD) analysis. This confirmed that in the presence of water, ammonium bicarbonate was collected (possibly in equilibrium with ammonium carbonate) while in the absence of water, the solid was predominantly ammonium carbonate (FIG. 5).

Without being bound by theory, it is believed that small amounts of ammonium carbamate will also be present, and that each of ammonium carbonate, ammonium bicarbonate and ammonium carbamate are present in equilibrium with each other in a solid or aqueous solution.

The recyclability of ammonia and carbon dioxide from the gas-liquid separator was confirmed by heating the aqueous reactor condensate up to 60° C., at which point the aqueous ammonium and carbonate/bicarbonate ions fully decomposed to release NH₃ and CO₂ gas, leaving water behind.

1. A process for producing one or more reaction products, the process comprising:
(i) providing ammonia and carbon dioxide to a reactor comprising a transition metal catalyst; and
(ii) reacting the ammonia and carbon dioxide in the presence of the transition metal catalyst to form one or more reaction products selected from the group consisting of carbon monoxide, one or more C₁₋₃ hydrocarbons, and hydrogen,
wherein step (ii) is conducted in a single reactor.

2. The process according to claim 1, wherein the transition metal catalyst comprises a transition metal from one or more of Groups 8 to 11 of the periodic table.

3. The process according to claim 1, wherein the transition metal catalyst comprises one or more transition metals selected from the group consisting of Co, Fe, Cu, Ni, Ru and Pt.

4. The process according to claim 1, wherein the transition metal catalyst is in the form of a solid metal catalyst.

5. The process according to claim 1, wherein the transition metal catalyst is in the form of a solid metal catalyst provided on a solid support.

6. The process according to claim 5, wherein the solid support comprises one or more of the group consisting of silica, alumina, carbon, ceria, zirconia, gallium oxide, indium oxide, and magnesium oxide.

7. The process according to claim 1, wherein the transition metal catalyst is obtainable by a process comprising:
(a) co-precipitation of a transition metal salt and a solid support precursor; and
(b) calcining or reducing the resulting product

8. The process according to claim 5, wherein the transition metal catalyst comprises from 1 wt. % to 70 wt. % transition metal.

9. The process according to claim 1, wherein the molar ratio of ammonia:carbon dioxide is from about 0.67:1 to about 10:1.

10. The process according to claim 1, wherein step (ii) is conducted at a temperature of from about 250° C. to about 1000° C.

11. The process according to claim 10, wherein step (ii) is conducted at pressure of from atmospheric pressure to about 3,000 kPa.

12. The process according to claim 1, the process further comprising:
(iii) cooling the output products from step (ii) in a gas-liquid separator to provide a gaseous stream comprising one or more of carbon monoxide, one or more C₁₋₃ hydrocarbons, and hydrogen.

13. The process according to claim 12, wherein step (iii) is conducted at a temperature of from about 1° C. to about 40° C.

14. The process according to claim 12, wherein step (iii) also provides a liquid stream comprising one or more of ammonium carbonate, ammonium bicarbonate and ammonium carbamate, and where the process further comprises:
(iv) passing the liquid stream comprising one or more of ammonium carbonate, ammonium bicarbonate and ammonium carbamate through an evaporator to regenerate ammonia and carbon dioxide.

15. The process according to claim 14, wherein step (iv) is conducted at a temperature of from about 40° C. to about 80° C., such as about 60° C.

16. The process according to claim 1, wherein step (ii) is conducted in an atmosphere comprising less than 1 vol. % O₂.

17. The process according to claim 1, comprising one or both of the following precursor steps:
(A) obtaining a gaseous stream comprising carbon dioxide from a carbon capture process or from a stock of liquid carbon dioxide; and
(B) obtaining a gaseous stream comprising ammonia from a stock of liquid ammonia.

18. (canceled)

19. (canceled)

20. The process according to claim 1, wherein:
the one or more C₁₋₃ hydrocarbons comprises methane;
the transition metal catalyst comprises one or more transition metals selected from the group consisting of Co, Fe, Cu, Ni, Ru and Pt in the form of a solid metal catalyst provided on a solid support; and
step (ii) is conducted at a temperature of from about 300° C. to about 700° C.

21. The process according to claim 20, the process further comprising:
(iii) cooling the output products from step (ii) in a gas-liquid separator to provide a gaseous stream comprising one or more of carbon monoxide, one or more C₁₋₃ hydrocarbons, and hydrogen, and a liquid stream comprising one or more of ammonium carbonate, ammonium bicarbonate and ammonium carbamate; and
(iv) passing the liquid stream comprising one or more of ammonium carbonate, ammonium bicarbonate and ammonium carbamate through an evaporator to regenerate ammonia and carbon dioxide.

22. (canceled)

23. An apparatus for conducting the process according to claim 1, wherein the apparatus comprises:
(a) a carbon dioxide storage tank for storing carbon dioxide in liquid form;
(b) an ammonia storage tank for storing ammonia in liquid form;
(c) a packed bed catalytic reactor comprising a transition metal catalyst in the form of a solid metal provided on a solid support, said packed bed catalytic reactor being suitable for converting carbon dioxide and ammonia into one or more of carbon monoxide and methane;
(d) a thermal energy source for heating the packed bed catalytic reactor;
(e) a gas-liquid separator; and
(f) an evaporator.
